(19) United States
(12) Patent Application Publication
(10) Pub. No.: US 2009/0209922 A1
(43) Pub. Date: Aug. 20, 2009

(54) Self-Lubricating Elastomeric Components for Use in Medical Devices

(75) Inventor: Richard T. Boisjoly, Nashua, NH (US)

Correspondence Address: BROMBERG & SUNSTEIN LLP, 125 SUMMER STREET, BOSTON, MA, 02110-1618, US

(73) Assignee: NYPRO INC., Clinton, MA (US)

(21) Appl. No.: 12/371,185

(22) Filed: Feb. 13, 2009

Related U.S. Application Data
(60) Provisional application No. 61/028,933, filed on Feb. 15, 2008.

(51) Int. Cl. A61M 39/22; C10M 101/04; C08K 5/20; C10M 133/16

(52) U.S. Cl. 604/256; 508/100; 508/107; 524/230; 508/459

(57) ABSTRACT

A device includes an elastomer component that has a molecular scaffold. The molecular scaffold defines interstitial spaces. The component is impregnated with a vegetable oil carrying a fatty acid amide to impart a lubrication to the component and thereby impart a resistance to adhering to a surface of the component. In an illustrative device, a valve includes an elastomeric member that includes a vegetable oil and a fatty acid amide. A method for manufacturing such a device and a precursor for use in manufacturing are also disclosed.

### PRIORITY

This application for United States Patent claims priority to U.S. Provisional Patent Application Ser. No. 61/028,933, filed Feb. 15, 2008, entitled “Self-Lubricating Elastomeric Components for Use in Medical Devices”, which is hereby incorporated by reference in its entirety.

### TECHNICAL FIELD

The present invention relates to self-lubricating elastomeric components for use in medical devices.

### BACKGROUND

Certain medical devices include elastomeric components, which provide flexibility, resilience or softness to these components. For example, silicone may be molded to form an elastomeric component.

Medical valving devices often act as a sealed port that may be repeatedly accessed to non-invasively inject fluid into, or withdraw fluid from, a patient's vasculature. During use, medical personnel may insert a syringe into the proximal port of a properly secured medical valve to inject fluid into, or withdraw fluid from, a patient. Once inserted, the syringe may be used to freely inject or withdraw fluid to and from the patient. A medical valve with elastomeric components is disclosed in U.S. Pat. No. 6,039,302, for “Swabbable Luer-Activated Valve,” issued Mar. 21, 2001 to Andrew L. Cote, Sr. and Charles F. Ganem.

Unfortunately, there is a potential for elastomeric components to adhere to surfaces, especially other chemically similar surfaces of the same or other components. Self-adherence (i.e., “sticking”) may be problematic during the manufacturing process and thereby increase the cost of the components. In the context of an elastomeric gland of a medical valve, surfaces of a slit or gland may adhere to nearby surfaces. Such adherence is inconvenient because it requires swapping-out of the valving device, which can increase expense and delay medical care.

Lubricants have been used to prevent adhering of elastomeric components. Application of a lubricant to the surface of an elastomeric component is potentially difficult, costly, and non-reproducible. Additionally, lubricant may be absorbed by the elastomer and, as a result, unavailable to the surface during use.

### SUMMARY OF THE INVENTION

In accordance with an illustrative embodiment of the invention, a device includes an elastomer component that has a molecular scaffold. The molecular scaffold defines interstitial spaces. The component is impregnated with a vegetable oil carrying a fatty acid amide to impart a lubrication to the component and thereby impart a resistance to adhering to a surface of the component.

In related embodiments, due to the lubrication, a self-adhesion of the component is less than the adhesion of a similar component without the vegetable oil carrying the fatty acid amide. The fatty acid amid may impart the lubrication. The lubrication may be imparted by a migration of fatty acid amide to the surface. The vegetable oil and fatty acid amide may be selected to cause a rate and duration of the migration so that the component is in a self-lubricated state when used prior to a product expiration date. The apparatus may be a medical valve with a gland component that includes the elastomeric polymer. The device may be a medical valve with a gland that comprises the elastomeric polymer.

In other related embodiments, the fatty acid amide may be oleamide or erucamide. The concentration of the fatty acid amide maybe between 0.01% and 2%. The elastomer may comprise a silicone polymer.

In a related embodiment, the fatty acid amide is not in crystallized or aggregated into potentially hazardous particles.

In accordance with another illustrative embodiment, an elastomeric precursor includes a polymer precursor that is capable of being polymerized into an elastomeric polymer; a vegetable oil; and a fatty acid amide.

In related embodiments, the fatty acid amide may be oleamide or erucamide. The vegetable oil and fatty acid amide may be selected to cause a migration of fatty acid amide to a surface of a component formed from polymerization of the precursor to be in a self-lubricated state.

In accordance with yet another embodiment of the present invention a method for manufacturing an elastomeric component includes combining a polymer precursor capable of being polymerized into an elastomeric polymer, a vegetable oil, and a fatty acid amide. The precursor is polymerized to create an elastomer that is impregnated with the oil having the fatty acid amide dissolved therein.

In related embodiments, the fatty acid amide may be dissolved in the oil. The combined precursor, oil and fatty acid amide may be introduced into a mold prior to polymerizing the monomer. The vegetable oil may be heated to dissolve the fatty acid amide. The precursor, oil, and amide may be chosen to cause a rate and duration of migration to a surface of the elastomer that causes the elastomer to be in a self-lubricated state when used prior to a product expiration date.

In a further embodiment, an apparatus is produced by combining a polymer precursor capable of being polymerized into an elastomeric polymer, a vegetable oil, and a fatty acid amide. The precursor is polymerized to create an elastomer that is impregnated with the oil having the fatty acid amide dissolved therein.

In accordance with a further embodiment of the invention, a valve includes a housing defining a passageway having an inlet and an outlet, the housing being substantially rigid, an actuation mechanism for toggling between open and closed modes of the valve, and a substantially flexible, resilient gland member having seal section with a normally closed aperture therethrough. The gland member includes an elastomeric member that includes a vegetable oil and a fatty acid amide.

In related embodiments, the vegetable oil and fatty acid amide may be selected so as to cause a migration of fatty amide to a surface of the elastomeric polymer and thereby cause the gland to be in a self-lubricated state. The fatty acid amide may be erucamide or oleamide. In a further related embodiment, the fatty acid amide does not form potentially hazardous particles.

### BRIEF DESCRIPTION OF THE DRAWINGS

The foregoing features of the invention will be more readily understood by reference to the following detailed description, taken with reference to the accompanying drawings, in which:

FIG. 1 shows a flow diagram of a method for producing a self-lubricating component in accordance with an embodiment of the invention; and

FIGS. 2A-2E show a longitudinal sectional view of a valve at various stages of actuation according to another embodiment of the invention.

### DETAILED DESCRIPTION OF SPECIFIC EMBODIMENTS

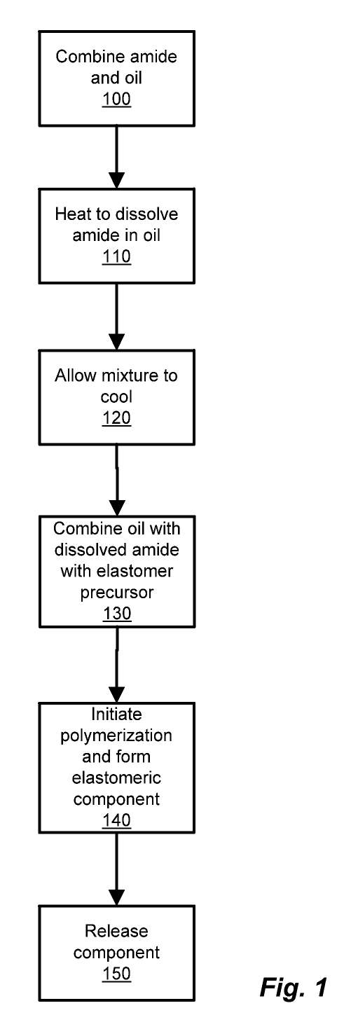

FIG. 1 shows a flow diagram of a method for producing a self-lubricating component in accordance with an embodiment of the invention. As used herein, a “component” may be an entire device or a part of a larger assembly. The process begins by combining a non-toxic fatty acid amide lubricant with an appropriately compatible, nontoxic oil (step 100). Suitable fatty acid amides include oleamide and erucamide and suitable oils include vegetable oils, such as canola.

Fatty acid amides such as oleamide and erucamide are typically available as powders that are insoluble in the precursors of elastomeric materials. Thus, if fatty acid amides are included in the process of casting an elastomeric component, residual particles may migrate to the surface of the component, producing a potentially hazardous state. For example, if incorporated into a gland of a medical valve, these particles may migrate to the surface of the gland and enter the vasculature of a patient, causing injury. Accordingly, illustrative embodiments of the invention include formulation methods that allow fatty acid amides to be solubilized, dissolved, or otherwise carried and incorporated into elastomeric compositions that are used in components. Additionally, specific embodiments may prevent the formation of particles of lubricant material in the elastomeric components.

In specific illustrative embodiments, a fatty acid amide is dissolved in an appropriate solvent. The solvent may be nontoxic; for example a vegetable oil, such as canola, soy, corn, or epoxidized oil (e.g. epoxidized soybean or linseed oil) may be used. If the amide and oil are mixed at low temperatures, a suspension may be formed. However, heating the combination will allow the amide to dissolve (step 110). Stirring or agitation may increase the dissolution rate and uniformity. For example, erucamide may be dissolved in canola oil at a 1% (by weight, i.e., 1 g of amide per 100 ml of oil) concentration. Alternately, the amide may be added to pre-heated oil.

The oil may then be allowed to cool (step 120). As may be calculated using known techniques, the time required for cooling depends on the rate of heat exchange with the vessel holding the mixture and the scale of the batch (if performed in batch mode). The mixture may also be de-aerated to encourage robust and uniform polymerization.

The oil containing the dissolved fatty acid amide is then combined with an elastomer precursor liquid (step 130). For example, the precursor may be a silicone polymer capable of being crosslinked to form a silicone rubber (i.e., a polysiloxane). The precursor may also include a monomer. Additionally, other ingredients may also be included, such as initiators of polymerization, antioxidants, emulsifiers (e.g., bisteramide or lecithin), colorants, secondary solvents, additional lubricants and glycerol. These other ingredients may, in some instances, act to provide synergistic effects in producing self-lubrication of the final part.

The oil, fatty acid amide, and precursor may be mixed or otherwise combined prior to casting. Alternately, the dissolved fatty acid amide and elastomer precursor may be mixed on-line by combining streams of each material as part of an integrated processing system or scheme.

The elastomer component is formed by initiating polymerization (e.g., crosslinking) (step 140). As is known in the art, polymerization may be initiated, among other ways, by combining the elastomer precursor with a catalyst and heating. The combined fatty acid amide, oil, and polymer may be injected into a mold (i.e., injection molded) to impart a shape to the component. As the component is formed, the elastomer precursor will polymerize or cross-link, form a molecular scaffold with interstitial spaces that are impregnated with the amide-carrying oil. The result is a self-lubricating elastomeric component, typically resilient, impregnated with both oil and fatty acid amide.

In an embodiment, the component will be in a metastable condition, in that migration of the fatty acid amide to the surface of the component is both kinetically feasible and thermodynamically favored. In other words, the fatty acid amide may be partially incompatible with the solvent/elastomer system. Careful selection of the oil and fatty acid amide as well as the concentration of the fatty acid amide in the oil will result in a system in which the fatty acid amide does not aggregate or crystallize to form hazardous or potentially hazardous particles, yet provides sufficient lubrication of the formed component to prevent unwanted adhering. Thus, the so-formed elastomer component has a self-lubrication that imparts a resistance to adhering to various surfaces, including a resistance to self-adhesion of multiple surface regions of the component. Because of the self-lubrication due to the erucamide and oil, the resistance to self-adhesion of the component is less than the self-adhesion of a similar component without the oil carrying the fatty acid amide.

Use of erucamide will give a slow migration or “bleeding” of the lubricant to the surface, whereas oleamide will migrate more quickly. Slow and fast migrating lubricants such as these may also be combined to impart upon the component a self-lubrication that initiates quickly yet persists over long periods (e.g., longer than the recommended shelf life of a device made with the component). In a specific embodiment, an elastomeric component includes a lubricant and solvent system (e.g., vegetable oil) that causes migration of the lubricant to a surface of the component with a rate and duration that is selected maintain self-lubrication over a time period that exceeds a given expected or predetermined shelf life of a product. Such a combination should increase the probability that the product is in a self-lubricated state when used prior to a product expiration date selected based on the shelf life. For example, the selected shelf life may be six months, one year or more.

In an embodiment, the concentration of fatty acid amide in the final part is from between 0.01 to 2%.

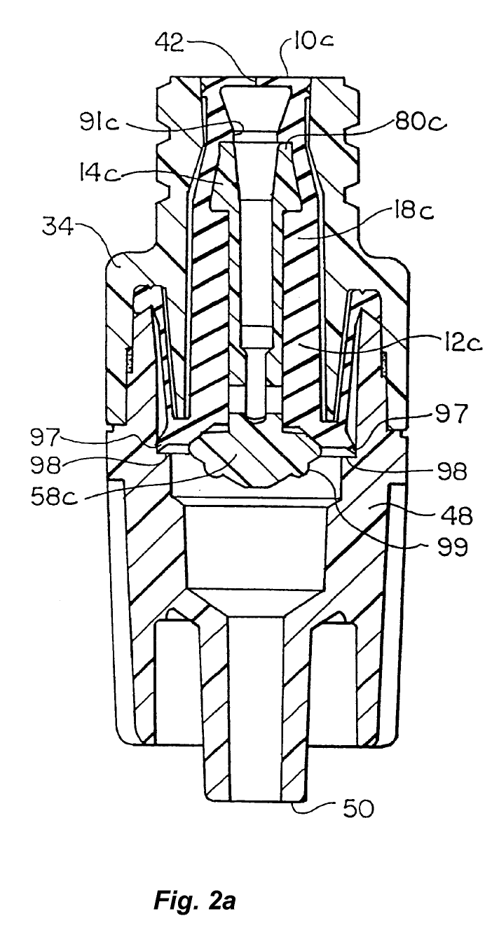

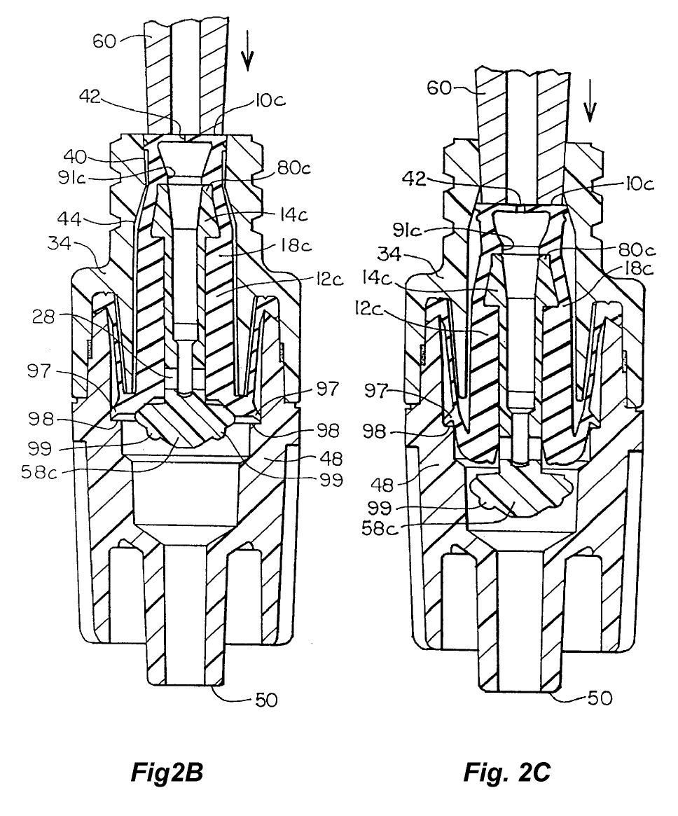

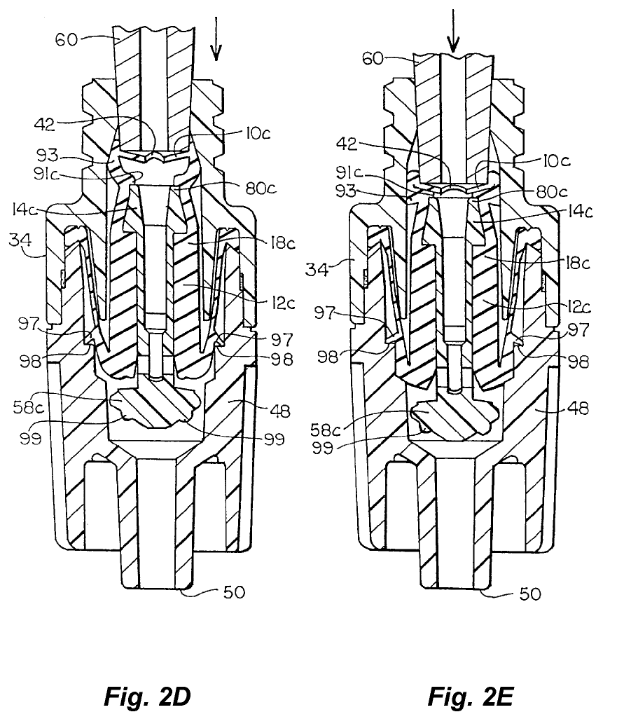

Illustrative embodiments may be used in a variety of different devices. Accordingly, FIG. 2a shows a self-lubricating medical valve in accordance with another embodiment of the invention. This valve is but one of a plurality of different types of valves that may incorporate illustrative embodiments of the invention. This valve includes a movable center cannula 14c, located inside a gland 12c. The gland 12c is formed according to the method of FIG. 1. Accordingly, surfaces of the gland 12c are self-lubricating and resist adhering to other surfaces of gland 12c. Gland 12c is in turn is located within the passageway formed by the inlet housing portion 34 and the outlet housing portion 48. When the valve is in the closed position, the gland's seal section 10c is spaced away from the top end 80c of the cannula 14c. When the valve is being opened, as shown in FIGS. 2A-2D, the gland's seal section 10c moves towards the cannula's top surface 80c. This movement is limited by a step 91c on the inner surface of the gland 12c, which prevents the seal section 10c from moving past cannula's top surface 80c.

The gland 12C of FIG. 2a includes a ridge 97 that normally is seated on a ledge 98 formed by the interior walls of the outlet housing portion 48. In addition, the tapered outlet end 58c of the cannula 14c includes ribs 99 for limiting longitudinal motion of the cannula 14c toward the outlet end 50 of the valve. Accordingly, there is no need for ribs to protrude from the interior walls of the outlet housing portion 48.

FIGS. 2B-2E show of the valve of FIG. 2 as it is urged by a luer-taper nozzle 60 from a substantially fully closed position to a substantially fully open position. Specifically, in FIG. 2B, the seal section 10c is substantially aligned with the exterior inlet face 52 and extends slightly beyond the exterior inlet face to provide a swabbable surface. The outer diameter of the seal section 10c is a little greater than the inner diameter of the inlet's tapered section 40, so that the resulting pressure keeps an aperture 42 closed when the valve is in the closed position. Because the valve includes the high-pressure seal area 22, the seal aperture 42 does not have to resist high back pressure.

As the nozzle 60 is inserted into the valve's inlet, as shown in FIG. 2C, the gland's seal section 10c is urged towards the cannula 14c, which in turn is urged towards the valve's outlet 50. As the seal section 10c moves from the inlet's tapered section 40 to the inlet's expanding section 44, which has a greater inner diameter than the seal section's outer diameter, the aperture 42 in the gland's seal section 10c begins to open. If opposing portions of aperture 42 were to adhere, the valve might be rendered unusable. However, the presence of lubricant on the surface of the aperture 42 prevents such unwanted adherence. Without wanting to be bound by the mechanism, the lubricant may form a boundary layer at the plane formed by the aperture 42.

The cannula's outlet end 58c begins to separate from the gland 12c, opening the high-pressure seal and providing fluid communication between the cannula's transverse passage 28 and the valve's outlet 50.

As the nozzle 60 is further inserted into the valve's inlet, as shown in FIG. 2D, the seal section 10c moves further in the inlet's expanding section 44, so that the increasing inner diameter of the inlet permits the seal section's aperture 42 to open further. The step 91c on the inner surface of the gland 12c is pressed against the top surface 80c of the cannula 14c, so that further movement of the seal section 10c towards the cannula 14c causes deformation of the sidewalls 93 of the gland 12c adjacent the seal section 10c.

The cannula's top surface 80c, along with the gland's step 91c, prevents the seal section 10c from being pushed beyond the cannula's top surface 80c, as shown in FIG. 2E. FIG. 2E shows the nozzle 60 fully inserted into the valve with the seal section's aperture 42 fully opened. By keeping the seal section 10c from being pushed beyond the cannula's top surface 80c, the seal section 10c is able to spring back to its original position quickly, when the nozzle is removed from the valve. Moreover, the ribs 99 on the outlet end 58c of the cannula 14c limit further longitudinal movement of the cannula 14c toward the outlet 50. It should be noted that the ridge 97 remains seated on the ledge 98 throughout the entire process shown in FIGS. 2B-2E.

Example: Kemamide® E erucamide (Crompton Corporation) is weighed out and added to 1 quart of canola oil to 1% by weight. Dissolution of the Kemamide E is encouraged by heating the oil to 250° F. The solution is stirred and de-aerated under vacuum for about 15 minutes and then allowed to cool at room temperature for 1 hour. The solution is labeled with batch and lot information and staged in a processing area. The stored solution is then transferred to the “3rd stream” additive chamber of an Elmet silicone metering unit (Elmet Elastomere Produktions GmbH, Vienna, Austria). The silicone metering unit is programmed to dispense a 2% solution using the color dispensing controls of the unit's user interface. The appropriate vale is activated to prepare the unit for processing. The Kemamide solution is dispensed into the mixing block. The Elmet unit pumps “A” and “B” liquid injection molding/liquid silicone rubber materials (LSR 4060; Momentive Performance Materials; Wilton, Conn.) into the mixing block. Mixture of the “A” and “B” liquids creates a mixture of polymer, catalyst, inhibitor and additives, which upon heating cross-links to create the elastomeric component. The polymer has a molecular weight of about 80,000 Da.

A static mixer blends the resultant in-line mixture. The mixture is conveyed through a series of valves and a filtration module into a throat of a molding machine. The mixture is further conveyed by reciprocating screw and injected into a Gland Mold, which is preheated to about 350° F. After the molding cycle is completed, the finished glands are ejected from the mold and the cycle is repeated.

Methods according to embodiments described herein may also be used in other applications including food service, pharmaceutical, medical devices, automotive, etc. The elastomers produced are not limited to silicone, but include urethane elastomers, polyester elastomers, and others.

The described embodiments of the invention are intended to be merely exemplary and numerous variations and modifications will be apparent to those skilled in the art. All such variations and modifications are intended to be within the scope of the present invention as defined in the appended claims.

What is claimed is:

1. A device comprising:
an elastomer component having a molecular scaffold defining interstitial spaces, the component impregnated with a vegetable oil carrying a fatty acid amide so as to impart a lubrication to the component and thereby impart a resistance to adhering to a surface of the component.

2. A component according to claim 1, wherein, due to the lubrication, a self-adhesion of the component is less than the adhesion of a similar component without the vegetable oil carrying the fatty acid amide.

3. A component according to claim 2, wherein the fatty acid amide imparts the lubrication.

4. A component according to claim 3, wherein migration of fatty acid amide to the surface imparts the lubrication.

5. A component according to claim 4, wherein the vegetable oil and fatty acid amide are selected so as to cause a rate and duration of the migration and thereby result in a component that is in a self-lubricated state when used prior to a product expiration date.

6. A component according to claim 1, wherein the device is a medical valve with a gland that comprises the elastomeric polymer.

7. A component according to claim 1, wherein the fatty acid amide is one of oleamide and erucamide.

8. A component according to claim 1, wherein the concentration of the fatty acid amide is between 0.01% to 2%.

9. A component according to claim 1, wherein the elastomer comprises a silicone polymer.

10. A component according to claim 1, wherein the fatty acid amide is not in crystallized or aggregated into potentially hazardous particles.

11. An elastomeric precursor mixture comprising:
a polymer precursor that is capable of being polymerized into an elastomeric polymer component;
a vegetable oil; and
a fatty acid amide.

12. An elastomeric precursor according to claim 11, wherein the fatty acid amide is oleamide or erucamide.

13. An elastomeric precursor according to claim 11, wherein the vegetable oil and fatty acid amide are selected so as to cause a migration of fatty amide to a surface of a component formed from polymerization of the precursor and thereby cause the component to be in a self-lubricated state.

14. An elastomeric precursor according to claim 13, wherein a rate and duration of the migration are selected to cause the component to be in a self-lubricated state when used prior to a product expiration date.

15. A method for manufacturing an elastomeric component, the method comprising;
combining a polymer precursor capable of being polymerized into an elastomeric 25 polymer with a vegetable oil and a fatty acid amide; and
polymerizing the precursor to create an elastomer that is impregnated with the oil having the fatty acid amide carried therein.

16. A method according to claim 15, wherein the fatty acid amide is dissolved in the oil.

17. A method according to claim 15, further comprising introducing the combined precursor, oil and fatty acid amide into a mold prior to polymerizing the monomer.

18. A method according to claim 15, further comprising heating the vegetable oil to dissolve the fatty acid amide.

19. A method according to claim 15, wherein the precursor, oil, and amide are chosen so as to cause a rate and duration of migration to a surface of the elastomer that causes the elastomer to be in a self-lubricated state when used prior to a product expiration date.

20. An elastomeric apparatus produced according to the method of claim 15.

21. A valve comprising:
a housing defining a passageway having an inlet and an outlet, the housing being substantially rigid;
an actuation mechanism for toggling between open and closed modes of the valve;
a substantially flexible, resilient gland having a seal section with a normally closed aperture therethrough,
the gland including an elastomeric polymer, the elastomeric polymer having a molecular scaffold defining interstitial spaces which are impregnated with a vegetable oil, the oil carrying a fatty acid amide.

22. A valve according to claim 21, wherein the vegetable oil and fatty acid amide are selected so as to cause a migration of fatty amide to a surface of the elastomeric polymer and thereby cause the gland to be in a self-lubricated state.

23. A valve according to claim 21, wherein the fatty acid amide is erucamide or oleamide.

24. A valve according to claim 21, wherein the fatty acid amide does not form potentially hazardous particles.
